(19) United States
(12) Patent Application Publication
(10) Pub. No.: US 2024/0009012 A1
(43) Pub. Date: Jan. 11, 2024

(54) Stent Delivery System

(71) Applicant: Boston Scientific Scimed, Inc., Maple Grove, MN (US)

(72) Inventors: Derek Kenneth Larson, Golden Valley, MN (US); Michael William Nagel, Andover, MN (US); Wade Michael Felska, Maple Grove, MN (US)

(73) Assignee: Boston Scientific Scimed, Inc., Maple Grove, MN (US)

(21) Appl. No.: 18/219,478

(22) Filed: Jul. 7, 2023

Related U.S. Application Data
(60) Provisional application No. 63/359,409, filed on Jul. 8, 2022.

(51) Int. Cl. A61F 2/95; A61F 2/966

(52) U.S. Cl. CPC A61F 2/9517; A61F 2/966; A61F 2/9524

(57) ABSTRACT

A stent delivery system is adapted to deliver and release a self-expanding stent with improved force transmission efficiency and reduced energy loss. The stent delivery system includes an inner member including a stent receiving region, a proximal member that includes a buckle-reducing member that is adapted to reduce collapsibility within the proximal member, and a deployment sheath that extends coaxially about the inner member and the proximal member and is movable between a position in which the deployment sheath constrains the self-expanding stent and a position in which the deployment sheath does not constrain self-expanding stent, the deployment sheath including a stretch-reducing member that is adapted to reduce stretchability within the deployment sheath, wherein a reduced collapsibility of the proximal member and a reduced stretchability of the deployment sheath together reduce energy loss within the stent delivery system.

### CROSS-REFERENCE TO RELATED APPLICATIONS

This application claims the benefit of priority under 35 U.S.C. § 119 of U.S. Provisional Application No. 63/359,409, filed Jul. 8, 2022, the entire disclosure of which is hereby incorporated by reference.

### TECHNICAL FIELD

The present invention pertains to medical devices and methods for making and using medical devices. More particularly, the present invention pertains to stent delivery systems.

### BACKGROUND

A wide variety of intracorporeal medical devices have been developed for medical use, for example, intravascular use. Some of these devices include stent delivery systems. These devices are manufactured by any one of a variety of different manufacturing methods and may be used according to any one of a variety of methods. Of the known stent delivery devices and methods for making and using the same, each has certain advantages and disadvantages. There is an ongoing need to provide alternative stent delivery devices as well as alternative methods for making and using stent delivery devices.

### BRIEF SUMMARY

The invention provides design, material, manufacturing method, and use alternatives for stent delivery systems including self-expanding stent delivery systems, clip members for use with stent delivery systems, and methods for making and using the same.

An example may be found in a stent delivery system adapted to deliver and release a self-expanding stent with reduced energy loss within the stent delivery system. The stent delivery system includes an inner member including a stent receiving region adapted to accommodate a self-expanding stent therein. A proximal member extends coaxially about the inner member, the proximal member having a distal end terminating near the stent receiving region, the proximal member including a buckle-reducing member that is adapted to reduce collapsibility within the proximal member. A deployment sheath extends coaxially about the inner member and the proximal member, the deployment sheath movable between a distal position in which the deployment sheath constrains the self-expanding stent and a proximal position in which the self-expanding stent is no longer constrained by the deployment sheath, the deployment sheath including a stretch-reducing member that is adapted to reduce stretchability within the deployment sheath. A reduced collapsibility of the proximal member and a reduced stretchability of the deployment sheath together reduce energy loss within the stent delivery system.

Alternatively or additionally, the stent delivery system may further include a gear rack assembly coupled to the deployment sheath, and a handle coupled to the inner member and to the deployment sheath. The handle includes an actuation member that is coupled to the gear rack assembly so that actuation of the actuation member shifts the longitudinal position of the gear rack assembly and the deployment sheath relative to the stent receiving region.

Alternatively or additionally, the deployment sheath may include an inner polymeric layer and an outer polymeric layer, with the stretch-reducing member disposed between the inner polymeric layer and the outer polymeric layer.

Alternatively or additionally, the stretch-reducing member may include a first braided member.

Alternatively or additionally, the first braided member may be adapted to resist elongation in response to an applied tensile force.

Alternatively or additionally, the buckle-reducing member may be encapsulated within a polymeric layer.

Alternatively or additionally, the buckle-reducing member may extend a substantial fraction of a length of the proximal member.

Alternatively or additionally, the buckle-reducing member may include a second braided member.

Alternatively or additionally, the second braided member may be adapted to resist buckling in response to an applied compressive force.

Alternatively or additionally, the stent delivery may be adapted for delivering self-expanding stents.

Another example may be found in a stent delivery system adapted to deliver and release a self-expanding stent with improved force transmission efficiency within the stent delivery system. The stent delivery system includes an inner member including a stent receiving region adapted to accommodate a self-expanding stent therein. A proximal member extends coaxially about the inner member, the proximal member extending proximally from near the stent receiving region, the proximal member including a buckle-reducing member that is adapted to reduce collapsibility within the proximal member. A deployment sheath extends coaxially about the inner member and the proximal member, the deployment sheath movable between a distal position in which the deployment sheath constrains the self-expanding stent and a proximal position in which the self-expanding stent is no longer constrained by the deployment sheath, the deployment sheath including a stretch-reducing member that is adapted to reduce stretchability within the deployment sheath. A reduced collapsibility of the proximal member and the reduced stretchability of the deployment sheath together improve force transmission efficiency within the stent delivery system.

Alternatively or additionally, the deployment sheath may include an inner polymeric layer and an outer polymeric layer, with the stretch-reducing member disposed between the inner polymeric layer and the outer polymeric layer.

Alternatively or additionally, the stretch-reducing member may include a first braided member.

Alternatively or additionally, the buckle-reducing member may be encapsulated within a polymeric layer.

Alternatively or additionally, the buckle-reducing member may extend a substantial fraction of a length of the proximal member.

Alternatively or additionally, the buckle-reducing member may include a second braided member.

Another example may be found in a stent delivery system adapted to deliver and release a self-expanding stent, the stent delivery system. The stent delivery system includes an inner member including a stent receiving region adapted to accommodate a self-expanding stent therein. A proximal bumper extends coaxially about the inner member, the proximal bumper including a buckle-reducing braided member encapsulated within a polymeric layer. A deployment sheath extends coaxially about the inner member and the proximal member, the deployment sheath including a stretch-reducing braided member disposed between an inner polymeric layer and an outer polymeric layer, the deployment sheath movable between a distal position in which the deployment sheath constrains the self-expanding stent and a proximal position in which the self-expanding stent is no longer constrained by the deployment sheath.

Alternatively or additionally, the buckle-reducing braided member may be adapted to resist crumpling in response to an applied compressive force.

Alternatively or additionally, the stretch-reducing braided member may be adapted to resist elongation in response to an applied tensile force.

Alternatively or additionally, the stent delivery may be adapted for delivering peripheral stents.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial cross-sectional side view of an example stent delivery system;

FIG. 2 is a side view of a portion of the example stent delivery system shown in FIG. 1;

FIG. 3 is a side view of another portion of the example stent delivery system shown in FIG. 1;

FIG. 4 is a side view of another portion of the example stent delivery system shown in FIG. 1;

FIG. 5 is a cross-sectional view taken along line 5-5 of FIG. 4;

FIG. 6 is a side view of another portion of the example stent delivery system shown in FIG. 1;

FIG. 7 is a side view of another portion of the example stent delivery system shown in FIG. 1;

FIG. 8 is a cross-sectional view taken along line 8-8 of FIG. 7;

FIG. 9 is a side view of another portion of the example stent delivery system shown in FIG. 1;

FIG. 10 is a side view of another portion of the example stent delivery system shown in FIG. 1;

FIG. 11 is a side view of another portion of the example stent delivery system shown in FIG. 1.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

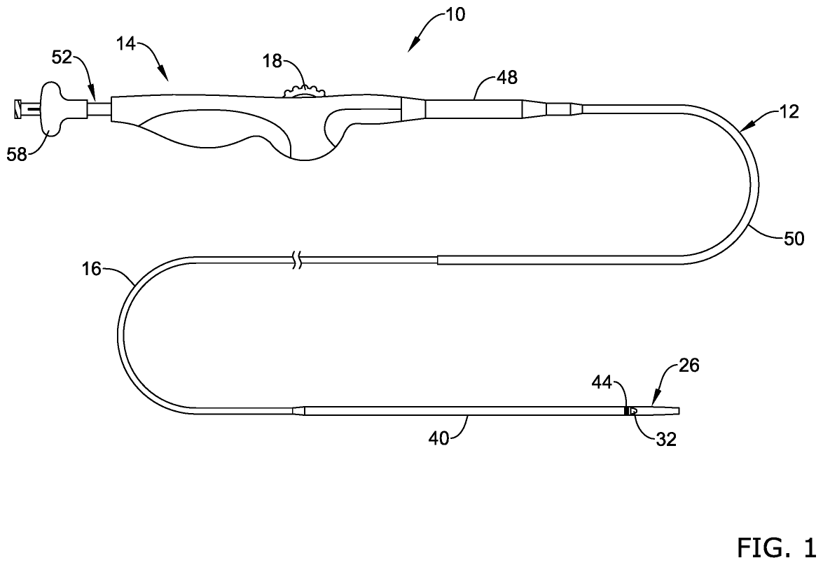

FIG. 1 illustrates an example stent delivery system 10. System 10 may include an elongate shaft 12 and a handle 14 coupled to shaft 12. In general, system 10 may be used to deliver a suitable stent, graft, endoprosthesis or the like to an area of interest within a body lumen of a patient. The body lumen may be a blood vessel located near the heart (e.g., within or near a cardiac vessel), within a peripheral vessel, within a neurological vessel, or at any other suitable location. In some cases, the stent delivery system 10 may include one or more features that reduce energy loss within the stent delivery system 10 while delivering a stent with the stent delivery system 10. These same one or more features may provide for improved force transmission through the stent delivery system 10.

It will be appreciated that improving force transmission through the stent delivery system 10 may provide for reduced energy loss within the stent delivery system 10 as the individual components within the stent delivery system 10 are adapted to perform their individual tasks while better maintaining the properties useful in delivering a stent. It will be appreciated that when delivering a stent via the stent delivery system 10, one or more components of the stent delivery system 10 may be placed in compression and one or more components of the stent delivery system 10 may be placed in tension. In some cases, it may be helpful to balance out particular properties of components subjected to compression and components subjected to tension.

In some instances, reducing energy loss may include reducing compression within components that are subjected to a compressive force and/or reducing elongation within components that are subjected to a tensile force. It will be appreciated that as a component subjected to a compressive force begins to buckle or compress, some of the energy that is applied to the stent delivery system 10 by a user applying a particular force, for example, goes into compressing the component, rather than achieving the desired effect on the component, which may for example include moving the component, or resisting movement if the component is functioning as a bumper, for example. As a component subjected to a tensile force begins to elongate, some of the of the energy that is applied to the stent delivery system 10 by a user applying a particular force, for example, goes into elongating the component, rather than achieving the desired effect on the component, which may for example include pulling the component away from another component.

Compression and/or elongation of components within the stent delivery system 10 cause energy loss because compression and/or elongation of components within the stent delivery system 10 utilize energy that would otherwise be applied to delivering a stent with the stent delivery system 10. As forces are applied to the stent delivery system 10, reducing the compression of any components that are subject to compressive forces means that less energy is wasted on compression of the component, and that as a result, force transmission efficiency improves. As forces are applied to the stent delivery system 10, reducing the elongation of any components that are subject to tensile forces means that less energy is wasted on elongation of the component, and that as a result, force transmission efficiency improves. It will be appreciated that force transmission efficiency provides an indication of how much of the force applied to the stent delivery system 10 actually goes to moving components of the stent delivery system 10 in order to deliver a stent, relative to how much of the applied force is lost to compression and/or elongation of components of the stent delivery system 10.

Reducing energy loss may be useful particularly when delivering stents such as self-expanding stents that have a relatively higher outward radial force. For example, some longer stents have a relatively higher outward radial force. Some stents having a large diameter have a relatively higher outward radial force. In some cases, a bare metal stent of particular dimensions may have a higher outward radial force relative to a stent having the same dimensions but including a coating on the stent, as the coating may alter possible frictional forces between the stent and the delivery device being used to deliver the stent. In some cases, peripheral stents may have a relatively high outward radial force.

Components that are placed in compression during delivery of a stent via the stent delivery system 10 may be adapted to resist length reductions as a response to an applied compressive force. As an example, a component that is placed in compression may be adapted to include, as will be discussed, a buckle-reducing member that helps to limit buckling, or length decreases, of the component including the buckle-reducing member.

Components that are placed in tension during delivery of a stent via the stent delivery system 10 may be adapted to resist stretching, or increases in length, as a result of an applied tensile force. As an example, a component that is placed in tension during delivery of a stent via the stent delivery system 10 may be adapted to include, as will be discussed, a stretch-reducing member that helps to limit stretching, or length increases, of the component including the stretch-reducing member.

Deployment of the stent may include the proximal retraction of a deployment sheath 16, which overlies the stent. Retraction of the deployment sheath 16 may include the actuation of an actuation member 18 generally disposed at the handle 14. In the example illustrated in FIG. 1, the actuation member 18 is a thumb wheel that can be rotated by a clinician in order to accomplish proximal retraction of the deployment sheath 16. Numerous other actuation members are contemplated. A number of other structures and features of the stent delivery system 10 can be seen in FIG. 1 and are labeled with reference numbers. Additional discussion of these structures can be found below.

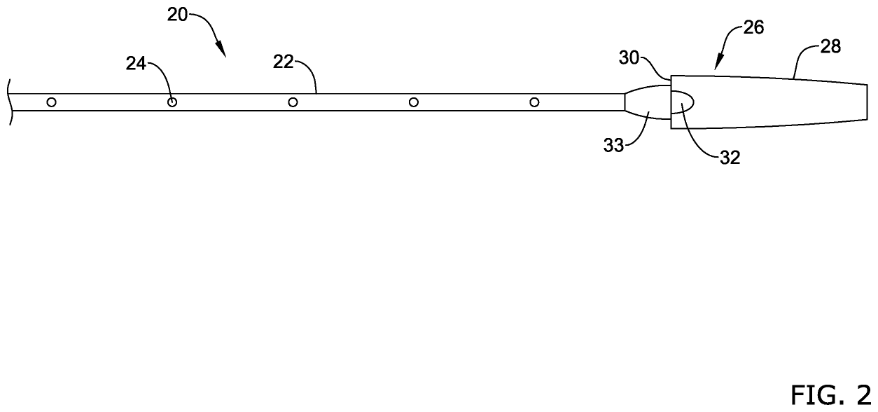

FIGS. 2-8 illustrate at least some of the structural components that may be included as a part of the stent delivery system 10. For example, the stent delivery system 10 may include an inner shaft or member 20 as illustrated in FIG. 2. In at least some embodiments, the inner member 20 may be a tubular structure and, thus, may include a lumen (not shown). The lumen may be a guidewire lumen that extends along at least a portion of the length of the inner member 20. Accordingly, the stent delivery system 10 may be advanced over a guidewire to the desired target location in the vasculature. In addition, or in alternative embodiments, the lumen may be a perfusion/aspiration lumen that allows portions, components, or all of the stent delivery system 10 to be flushed, perfused, aspirated, or the like.

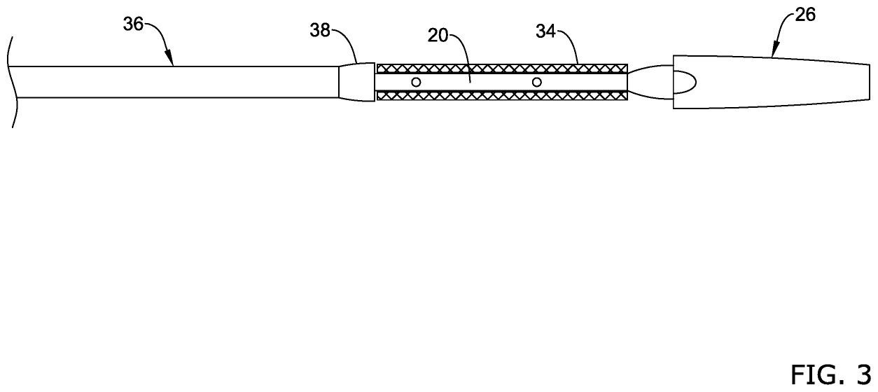

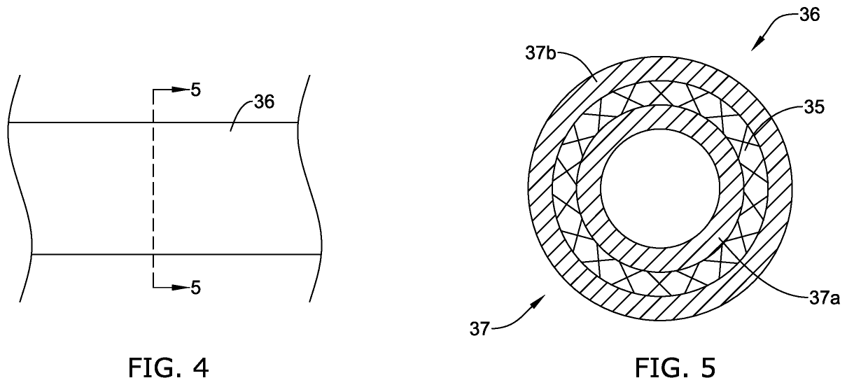

The inner member 20 may include a stent receiving region 22 about which a stent (not shown, can be seen in FIGS. 3-4) may be disposed. The length and/or configuration of stent receiving region 22 may vary. For example, stent receiving region 22 may have a length sufficient for the stent to be disposed thereon. It can be appreciated that as the length of the stent utilized for the stent delivery system 10 increases, the length of stent receiving region 22 also increases.

Along or otherwise disposed adjacent the stent receiving region 22 may be one or more perfusion ports 24. The perfusion ports 24 may extend through the wall of the inner member 20 such that fluid may be infused through the lumen of the inner member 20 and may be flushed through the ports 24. This may be desirable for a number of reasons. For example, the ports 24 may allow a clinician to evacuate air bubbles that may be trapped adjacent the stent by perfusing fluid through the ports 24. In addition, the ports 24 may be used to aspirate fluid that may be disposed along the inner member 20. The ports 24 may also aid in sterilization and/or other preparatory processing steps that may be involved in preparing the stent delivery system 10 for sale.

A tip 26 may be attached to or otherwise disposed at the distal end of the inner member 20. The tip 26 may generally have a rounded or smooth shape that provides a generally atraumatic distal end to the stent delivery system 10. For example, the tip 26 may have a smooth tapered distal portion 28 that gently tapers. The tip 26 may also include a proximal ridge 30 that is configured so that the deployment sheath 16 can abut therewith. The tip 26 may also include a tapered proximal portion 33. Numerous other shapes and/or configurations are contemplated for the tip 26.

The tip 26 may also include one or more cutouts or flats 32 formed therein. For the purposes of this disclosure, the flats 32 are understood to be cutouts or flattened portions of the tip 26 where the outer dimension or profile of the tip 26 is reduced. The name “flats” comes from the fact that these regions may have a somewhat “flat” appearance when compared to the remainder of the tip 26, which generally may have a rounded profile. The shape, however, of the flats 32 is not meant to be limited to being flat or planar as numerous shapes are contemplated.

The flats 32 may allow for a gap or space to be defined between the inner member 20 and the deployment sheath 16 when the deployment sheath 16 abuts the proximal ridge 30 of the tip 26. This gap may allow for fluid, for example perfusion fluid passed through the ports 24, to flow out from the deployment sheath 16. Thus, the flats 32 may be used in conjunction with the ports 24 to allow portions or all of the stent delivery system 10 to be flushed or otherwise evacuated of air bubbles.

FIG. 3 illustrates the inner member 20 with some additional structure of the stent delivery system 10. In this figure, a stent 34 is disposed about the inner member 20 (e.g., about the stent receiving region 22 of the inner member 20). In some instances, the stent 34 is a self-expanding stent. Accordingly, the stent 34 may be biased to outwardly expand. Because of this, the stent 34 may not be “loaded onto” the inner member 20 in a strict sense but rather may be thought of as being disposed about or surrounding the inner member 20. The stent 34 may then be restrained within the deployment sheath 16. In other cases, however, the stent 34 may be directly loaded onto the inner member 20 via crimping or any other suitable mechanical holding mechanism.

An intermediate tube 36 may also be disposed over the inner member 20. In at least some cases, the intermediate tube 36 may extend from a position adjacent to the proximal end of the inner member 20 to a position proximal of the distal end of the inner member 20. The intermediate tube 36 may include a bumper 38. In practice, the bumper 38 may function by preventing any unwanted proximal movement of the stent 34 during navigation and/or deployment of the stent 34.

The bumper 38 may have any suitable form. In some embodiments, the bumper 38 may be defined by a relatively short tube or sleeve that is disposed about the intermediate tube 36. The material utilized for the sleeve may be the same or different from that of the intermediate tube 36. The intermediate tube 36 may have a tapered or otherwise smooth transition in outer diameter adjacent the bumper 38. For example, polymeric material may be disposed or reflowed adjacent the bumper 38 (which may include disposing the polymeric material about a portion or all of the bumper 38) so as to define a gentle transition in outer diameter at the bumper 38. Other configurations are contemplated and may be utilized in alternative embodiments.

In some instances, the intermediate tube 36 may be placed in compression when delivering the stent 34 via the stent delivery system 10, as the intermediate tube 36 serves to locate the position of the bumper 38. As the deployment sheath 16 is withdrawn proximally in order to deploy the stent 34, it will be appreciated that the deployment sheath 16, or an interior surface thereof, may apply a force to the stent 34 as a result of frictional forces between the stent 34 and the interior surface of the deployment sheath 16. This applied force may urge the stent 34 proximally. The bumper 38, which as noted is located and positioned by the intermediate tube 36, serves to resist this applied force to the stent 34, allowing the stent 34 to remain in position.

In some cases, the intermediate tube 36 may include a buckle-reducing member that is adapted to resist buckling in response to applied compressive forces. FIG. 4 is a side view of a portion of the intermediate tube 36 and FIG. 5 is a cross-sectional view of the intermediate tube 36, taken along the line 5-5 of FIG. 4. The intermediate tube 36 includes a buckle-reducing member 35 that is encapsulated within a polymeric layer 37. In some cases, the buckle-reducing member 35 may have a buckle strength sufficient to resist buckling in response to forces applied to the intermediate tube 36 when withdrawing the deployment sheath 16 to deploy the stent 34. For example, withdrawing the deployment sheath 16 proximally can create frictional forces between the deployment sheath 16 and the stent 34, which can cause the stent 34 to deliver a compressive force to the intermediate tube 36 as the intermediate tube 36 resists proximal movement of the stent 34.

In some cases, as shown, the polymeric layer 37 may be considered as including an inner portion 37a and an outer portion 37b. In some cases, for example, the intermediate tube 36 may be formed by dip coating to form the inner portion 37a. The buckle-reducing member 35 may be disposed about the inner portion 37a, and then dip coating may continue to form the outer portion 37b, thereby encapsulating the buckle-reducing member 35 within the polymeric layer 37. Other ways of encapsulating the buckle-reducing member 35 within the polymeric layer 37 are also contemplated.

The buckle-reducing member 35 may be adapted to provide the intermediate tube 36 with an increased resistance to buckling, or to crumpling or otherwise undergoing a decrease in length of the intermediate tube 36 as a result of a compressive force being applied to the intermediate tube 36. The buckle-reducing member 35 may provide additional strength of the intermediate tube 36 without excessively impacting the flexibility of the intermediate tube 36 itself and thus not excessively impacting the flexibility of the stent delivery system 10 itself. In some cases, the polymeric layer 37, including the inner portion 37a and/or the outer portion 37b, may also contribute to improving buckle-resistance of the intermediate tube 36. In some instances, a resistance to buckling of the intermediate tube 36 may be considered as a summation of the buckle-resistance potentially provided by each of the components forming the intermediate tube 36.

The buckle-reducing member 35 may extend substantially an entire length of the intermediate tube 36. In some cases, the buckle-reducing member 35 may only extend a portion of the entire length of the intermediate tube 36. As an example, the buckle-reducing member 35 may be disposed within a portion of the intermediate tube 36 that extends within the handle 14. In some cases, the buckle-reducing member 35 may include two or more distinct sections of buckle-reducing member 35 that are each encapsulated within the polymeric layer 37, each section longitudinally spaced from another section.

The buckle-reducing member 35 may take any of a variety of different forms. In some cases, the buckle-reducing member 35 may be a slotted hypotube, for example. In some cases, the buckle-reducing member 35 may be a braided member such as a stainless steel braid flat wire ribbon. In some cases, the braided member may have a pic count that varies by length. The braided member may be formed of a flat wire or a ribbon wire. The braided member may be formed of a flat or ribbon wire that is formed of a polymeric material or a metallic material. The diameter of the wire or wires used to form the braided member may also vary.

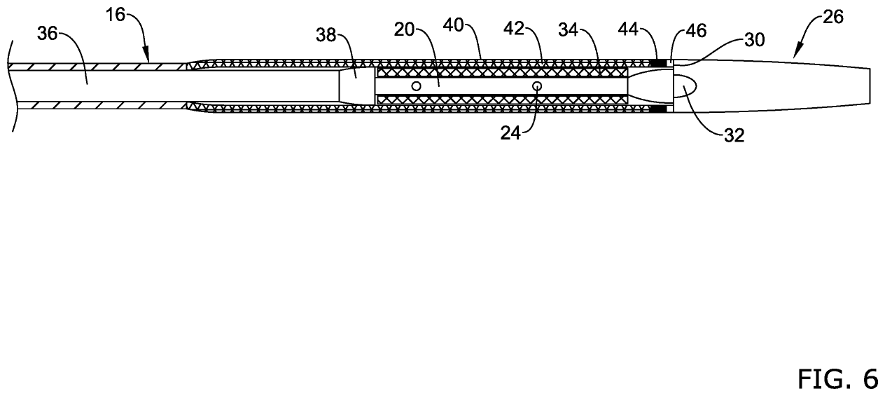

FIG. 6 illustrates additional structure of the stent delivery system 10. Here, the deployment sheath 16 can be seen disposed over the inner member 20, the intermediate tube 36, and the stent 34. It can be appreciated that the deployment sheath 16 is configured to shift between a first position, for example as shown in FIG. 6, where the deployment sheath 16 overlies the stent 34 and a second position where the deployment sheath 16 is proximally retracted to a position substantially proximal of the stent 34. In general, the first position may be utilized during navigation of the stent delivery system 10 to the appropriate location within a body lumen and the second position may be used to deploy the stent 34.

Deployment sheath 16 may include a flared portion 40 where the outer diameter of the deployment sheath 16 is increased. In portion 40, the thickness of the tubular wall of the deployment sheath 16 may or may not be increased. The flared portion 40 may be desirable for a number of reasons. For example, the flared portion 40 may allow the deployment sheath 16 to have an adequate inner dimension that is suitable so that the deployment sheath 16 may be disposed about the stent 34 and the bumper 38.

In some cases, the deployment sheath 16 may undergo a tensile force when the deployment sheath 16 is moved proximally as a result of frictional and other forces created between the stent 34 and the interior surface of the deployment sheath 16. In some cases, the deployment sheath 16 may include a stretch-reducing member 42 that is adapted to resist stretching in response to applied tensile forces. In some instances, the stretch-reducing member 42 may be adapted to resist elongation in response to applied forces. The stretch-reducing member 42 may be adapted to have sufficient strength to balance the strength of the buckle-reducing member 35.

The stretch-reducing member 42 may provide additional strength of the deployment sheath 16 without excessively impacting the flexibility of the deployment sheath 16 itself and thus not excessively impacting the flexibility of the stent delivery system 10. The stretch-reducing member 42 may take any of a variety of different forms. In some cases, the stretch-reducing member 42 may be a slotted hypotube, for example. In some cases, the stretch-reducing member 42 may be a braided member such as a stainless steel braid. For example, the stretch-reducing member 42 may include a braid, coil, mesh, combinations thereof, or the like, or any other suitable configuration. In some embodiments, the stretch-reducing member 42 may extend along the entire length of sheath 16. In other embodiments, the stretch-reducing member 42 may extend along one or more portions of the length of sheath 16. For example, the stretch-reducing member 42 may extend along the flared portion 40.

In some cases, the stretch-reducing member 42 may be a stainless steel braid having a total of 32 braid ribbons that are arranged in a 2×2 pattern. The stainless steel braid may have a low pic count and a high braid angle. In some cases, the stretch-reducing member 42 may include 32 individual ribbons that are configured in a 1×1 pattern and at a low braid angle to provide varied flexural properties. The stretch-reducing member 42 may also be configured as 16 wires in this pattern and braid angle but be substantially larger in ribbon size (2-3× of the previous example) to provide desired performance. In some cases, axially-aligned wires may be included within the braid pattern to further improve stretch resistance. This is or particular benefit for systems that require planarity in flexural performance. The exact design configuration is dependent on the end device's specific user-requirements for clinical performance and can be tuned to deliver the needed specifications. This may be done in concert with the other members of the stent delivery system 10 to accomplish the increased force transmission efficiency of the stent delivery system 10.

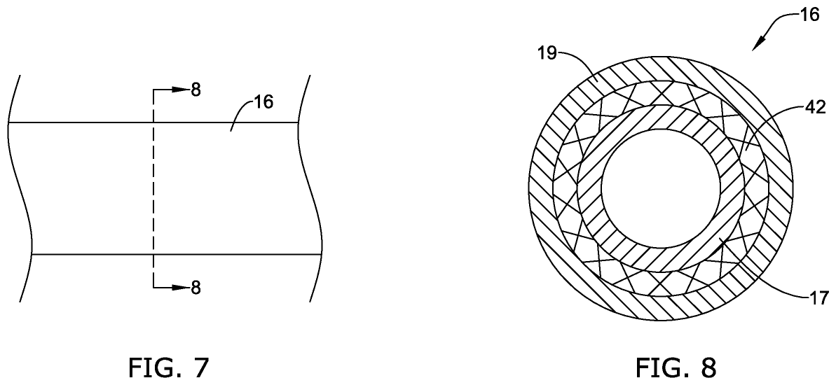

FIGS. 7 and 8 provide additional details regarding the deployment sheath 16. FIG. 7 is a side view of a portion of the deployment sheath 16 while FIG. 8 is a cross-sectional view of the deployment sheath 16 illustrating that the deployment sheath 16 may include an inner polymeric layer 17 and an outer polymeric layer 19, with the stretch-reducing member 42 disposed between the inner polymeric layer 17 and the outer polymeric layer 19. In some cases, the inner polymeric layer 17 and/or the outer polymeric layer 19 may contribute to a reduced stretchability of the deployment sheath 16. In some instances, a reduced stretchability of the deployment sheath 16 may be considered as a summation of the stretchability reductions potentially provided by each of the components forming the deployment sheath 16.

In some cases, the inner polymeric layer 17 may be a lubricious polymeric material such as but not limited to a perfluoro material. In some cases, the inner polymeric layer 17 may be PTFE (polytetrafluoroethylene). In some cases, the outer polymeric layer 19 may be formed of any suitable polymeric materials. As an example, the outer polymeric layer 19 may be formed of a nylon material such as that available from Vestamid. Pebax materials may also be used. The outer polymeric layer 19 may be cross-linked, for example. While the deployment sheath 16 is described as having the stretch-reducing member 42 disposed between the inner polymeric layer 17 and the outer polymeric layer 19, in some cases the deployment sheath 16 may include one or more additional layers. Each of the additional layers may be adapted to limit stretchability, for example.

Returning to FIG. 6, the deployment sheath 16 may also include a radiopaque marker or band 44. In general, the marker band 44 may be disposed adjacent to the distal end 46 of the deployment sheath 16. One or more additional marker bands 44 may be disposed along other portions of the deployment sheath 16 or other portions of the stent delivery system 10. The marker band 44 may allow the distal end 46 of the deployment sheath 16 to be fluoroscopically visualized during advancement of the stent delivery system 10 and/or deployment of the stent 34.

FIG. 6 also illustrates the distal end 46 of the deployment sheath 16 abutting the proximal ridge 30. In this configuration, the stent 34 can be flushed (e.g., to remove air bubbles) by infusing fluid through the inner member 20 and through the ports 24. Because of the flats 32, fluid may be allowed to be flushed out of the deployment sheath 16 by passing through the gaps formed between the inner member 20 and the deployment sheath 16 at the flats 32.

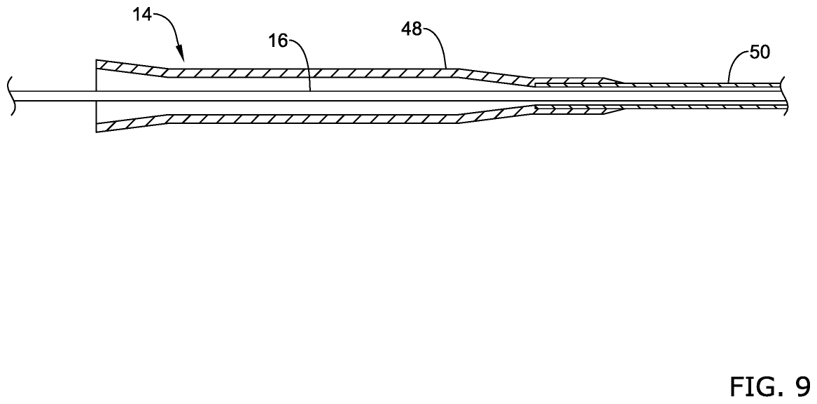

FIG. 9 illustrates a distal portion 48 of the handle 14. Here it can be seen that the handle 14 is attached to an outer member 50. The outer member 50 may be disposed about the deployment sheath 16 and extend along a portion of the length of the deployment sheath 16. Thus, along at least a portion of the length of the stent delivery system 10, the stent delivery system 10 may include four tubular structures that may be coaxially arranged—namely the outer member 50, the deployment sheath 16, the intermediate tube 36, and the inner member 20. In at least some embodiments, the outer member 50 may provide the stent delivery system 10 with a number of desirable benefits. For example, the outer member 50 may include or otherwise be formed from a lubricious material that can reduce friction that may be associated with proximally retracting the deployment sheath 16. In addition, the outer member 50 may include a surface that can be clamped or otherwise locked so that the position of stent delivery system 10 can be maintained without negatively impacting the retraction of the deployment sheath 16 (which might otherwise be impacted if the deployment sheath 16 was to be clamped). Numerous other desirable benefits may also be achieved through the use of the outer member 50.

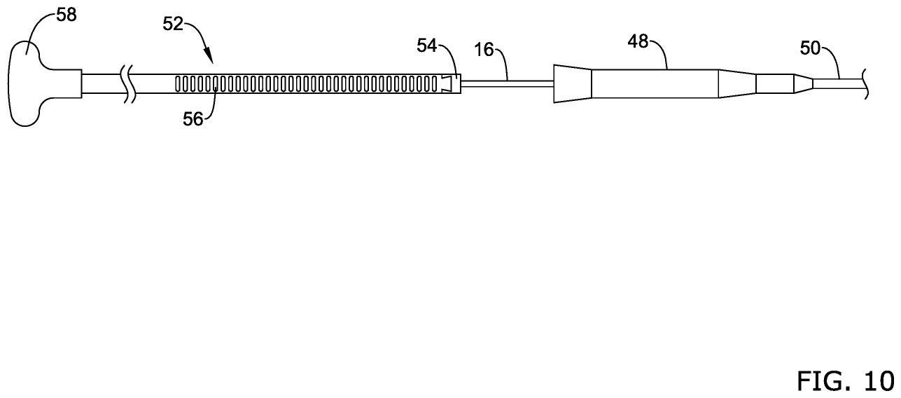

The deployment sheath 16 may pass proximally through the outer member 50 and extend proximally back within the handle 14. The intermediate tube 36 and the inner member 20 both also extend back within the handle 14 and are disposed within the deployment sheath 16. The proximal end of the deployment sheath 16 may be attached to a gear rack assembly 52 with a fastener or clip 54 as illustrated in FIG. 10. Thus, it can be appreciated that proximal movement of the gear rack assembly 52 may result in analogous proximal movement of the deployment sheath 16. The gear rack assembly 52 may include a plurality of teeth or gears 56. In practice, the teeth 56 may be configured to engage with corresponding teeth or gears (not shown) on the thumbwheel 18. Consequently, rotation of the thumbwheel 18, via gearing thereof with the gears 56, can be utilized to proximally retract the gear rack assembly 52 and, thus, the deployment sheath 16. Other structural arrangements may be utilized to accomplish proximal retraction of the gear rack assembly 52 through the actuation of the thumbwheel 18 or any other suitable actuation member.

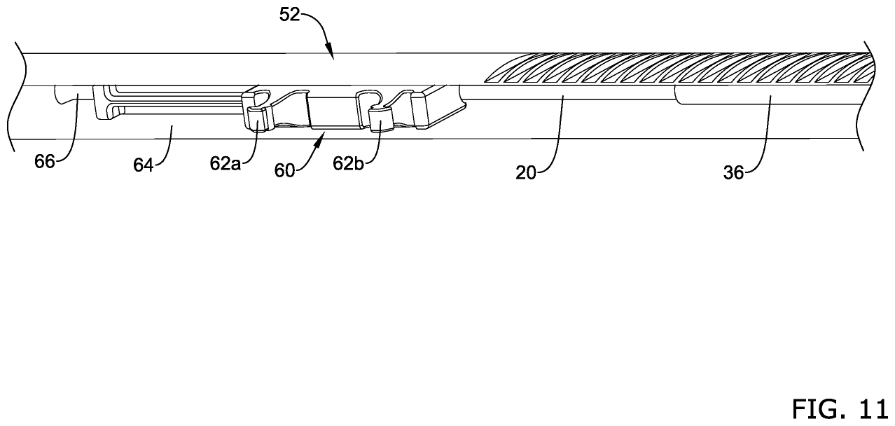

The gear rack assembly 52 may also include a flared proximal end 58. When properly assembly, the main body of the gear rack assembly 52 may be disposed within the handle 14 and the proximal end 58 may be disposed along the exterior of the handle 14. The gear rack assembly 52 may have a slot or groove 64 formed therein (not shown in FIG. 6, can be seen in FIG. 11). The groove 64 may extend the length of the gear rack assembly 52, including extending along the proximal end 58. Because the proximal end 58 may be generally located near the proximal end of the inner member 20, the flared shape of the proximal end 58 and the orientation of the groove 64 may allow the proximal end 58 to function as a guidewire introducer or funnel that may assist a clinician in placing, holding, removing, and/or exchanging a guidewire extending through the inner member 20.

The materials that can be used for the various components of the stent delivery system 10 (and/or other systems disclosed herein) may include those commonly associated with medical devices. For simplicity purposes, the following discussion makes reference to the shaft 12, the deployment sheath 16, and the inner member 20. However, this is not intended to limit the invention as the discussion may be applied to other similar members and/or components of members or systems disclosed herein.

The shaft 12, the deployment sheath 16, and the inner member 20, and/or other components of stent delivery system 10 may be made from a metal, metal alloy, polymer (some examples of which are disclosed below), a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof, and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the shaft 12, the deployment sheath 16, and the inner member 20 may also be doped with, made of, or otherwise include a radiopaque material including those listed herein or other suitable radiopaque materials.

In some embodiments, a degree of MRI compatibility is imparted into the stent delivery system 10. For example, to enhance compatibility with Magnetic Resonance Imaging (MRI) machines, it may be desirable to make the shaft 12, the deployment sheath 16, and the inner member 20, in a manner that would impart a degree of MRI compatibility. For example, the shaft 12, the deployment sheath 16, and the inner member 20, or portions thereof, may be made of a material that does not substantially distort the image and create substantial artifacts (artifacts are gaps in the image). Certain ferromagnetic materials, for example, may not be suitable because they may create artifacts in an MRI image. The shaft 12, the deployment sheath 16, and the inner member 20, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers that may be used to form the shaft 12, the deployment sheath 16, and the inner member 20, and/or other components of stent delivery system 10 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6% LCP.

In some embodiments, the exterior surface of the stent delivery system 10 may include a coating, for example a lubricious, a hydrophilic, a protective, or other type of coating. Hydrophobic coatings such as fluoropolymers provide a dry lubricity which improves device handling and exchanges. Lubricious coatings improve steerability and improve lesion crossing capability. Suitable lubricious polymers may include silicone and the like, polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, the entire disclosures of which are incorporated herein by reference.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A stent delivery system adapted to deliver and release a self-expanding stent with reduced energy loss within the stent delivery system, the stent delivery system, comprising:
an inner member including a stent receiving region adapted to accommodate a self-expanding stent therein;
a proximal member extending coaxially about the inner member, the proximal member having a distal end terminating near the stent receiving region, the proximal member including a buckle-reducing member that is adapted to reduce collapsibility within the proximal member;
a deployment sheath extending coaxially about the inner member and the proximal member, the deployment sheath movable between a distal position in which the deployment sheath constrains the self-expanding stent and a proximal position in which the self-expanding stent is no longer constrained by the deployment sheath, the deployment sheath including a stretch-reducing member that is adapted to reduce stretchability within the deployment sheath;
wherein a reduced collapsibility of the proximal member and a reduced stretchability of the deployment sheath together reduce energy loss within the stent delivery system.

2. The stent delivery system of claim 1, further comprising:
a gear rack assembly coupled to the deployment sheath; and
a handle coupled to the inner member and to the deployment sheath;
wherein the handle includes an actuation member, the actuation member being coupled to the gear rack assembly so that actuation of the actuation member shifts the longitudinal position of the gear rack assembly and the deployment sheath relative to the stent receiving region.

3. The stent delivery system of claim 1, wherein the deployment sheath comprises:
an inner polymeric layer and an outer polymeric layer, with the stretch-reducing member disposed between the inner polymeric layer and the outer polymeric layer.

4. The stent delivery system of claim 3, wherein the stretch-reducing member comprises a first braided member.

5. The stent delivery system of claim 4, wherein the first braided member is adapted to resist elongation in response to an applied tensile force.

6. The stent delivery system of claim 1, wherein the buckle-reducing member is encapsulated within a polymeric layer.

7. The stent delivery system of claim 6, wherein the buckle-reducing member extends a substantial fraction of a length of the proximal member.

8. The stent delivery system of claim 6, wherein the buckle-reducing member comprises a second braided member.

9. The stent delivery system of claim 8, wherein the second braided member is adapted to resist buckling in response to an applied compressive force.

10. The stent delivery system of claim 1, wherein the stent delivery is adapted for delivering self-expanding stents.

11. A stent delivery system adapted to deliver and release a self-expanding stent with improved force transmission efficiency within the stent delivery system, the stent delivery system, comprising:
an inner member including a stent receiving region adapted to accommodate a self-expanding stent therein;
a proximal member extending coaxially about the inner member, the proximal member extending proximally from near the stent receiving region, the proximal member including a buckle-reducing member that is adapted to reduce collapsibility within the proximal member;
a deployment sheath extending coaxially about the inner member and the proximal member, the deployment sheath movable between a distal position in which the deployment sheath constrains the self-expanding stent and a proximal position in which the self-expanding stent is no longer constrained by the deployment sheath, the deployment sheath including a stretch-reducing member that is adapted to reduce stretchability within the deployment sheath;
wherein a reduced collapsibility of the proximal member and the reduced stretchability of the deployment sheath together improve force transmission efficiency within the stent delivery system.

12. The stent delivery system of claim 11, wherein the deployment sheath comprises an inner polymeric layer and an outer polymeric layer, with the stretch-reducing member disposed between the inner polymeric layer and the outer polymeric layer.

13. The stent delivery system of claim 12, wherein the stretch-reducing member comprises a first braided member.

14. The stent delivery system of claim 11, wherein the buckle-reducing member is encapsulated within a polymeric layer.

15. The stent delivery system of claim 14, wherein the buckle-reducing member extends a substantial fraction of a length of the proximal member.

16. The stent delivery system of claim 15, wherein the buckle-reducing member comprises a second braided member.

17. A stent delivery system adapted to deliver and release a self-expanding stent, the stent delivery system, comprising:
an inner member including a stent receiving region adapted to accommodate a self-expanding stent therein;
a proximal bumper extending coaxially about the inner member, the proximal bumper including a buckle-reducing braided member encapsulated within a polymeric layer; and
a deployment sheath extending coaxially about the inner member and the proximal member, the deployment sheath including a stretch-reducing braided member disposed between an inner polymeric layer and an outer polymeric layer, the deployment sheath movable between a distal position in which the deployment sheath constrains the self-expanding stent and a proximal position in which the self-expanding stent is no longer constrained by the deployment sheath.

18. The stent delivery system of claim 17, wherein the buckle-reducing braided member is adapted to resist crumpling in response to an applied compressive force.

19. The stent delivery system of claim 17, wherein the stretch-reducing braided member is adapted to resist elongation in response to an applied tensile force.

20. The stent delivery system of claim 17, wherein the stent delivery is adapted for delivering peripheral stents.
